(19) United States
(12) Patent Application Publication
(10) Pub. No.: US 2026/0102551 A1
(43) Pub. Date: Apr. 16, 2026

(54) COLLECTION CANISTER AND QUICK RELEASE ADAPTER ASSEMBLY FOR VACUUM WITHDRAWAL SYSTEM

(71) Applicant: Precision Medical, Inc., Northampton, PA (US)

(72) Inventors: Michael Krupa, Northampton, PA (US); Blair Hollshwandner, Northampton, PA (US)

(73) Assignee: Precision Medical, Inc., Northampton, PA (US)

(21) Appl. No.: 19/357,754

(22) Filed: Oct. 14, 2025

Related U.S. Application Data
(60) Provisional application No. 63/706,245, filed on Oct. 11, 2024.

(51) Int. Cl. A61M 1/00

(52) U.S. Cl. CPC A61M 1/784; A61M 1/60; A61M 1/74; A61M 1/782

(57) ABSTRACT

A multi-function discardable collection canister for use with a vacuum system for withdrawing fluids from a patient site. The collection canister includes a housing positioned upstream of a vacuum regulator which is fluidly coupled to a vacuum source. A float valve positioned within the housing is normally in an open position to permit the withdrawn fluids to collect within the housing. When the fluids reach a predetermined threshold, the float valve moves to a closed position to prevent additional fluids from passing through the housing. A flow indicator indicates the presence of vacuum flow within the housing. A filter positioned within the housing impedes solids, liquids, and airborne particles from passing through the housing, which prevents damage to, and contamination of, the vacuum regulator and the vacuum source.

### BACKGROUND

The present invention relates generally to vacuum withdrawal systems used in hospital settings to withdraw fluids from a patient, and in particular to a collection canister arranged for use in combination with such systems.

Hospitals and other medical facilities typically have a central vacuum source on site and a series of lines leading from the central vacuum source to various operating rooms and patient rooms throughout the facility. A vacuum regulator is attached to the central vacuum source, typically at a wall connection, to provide a regulated source of suction. Specifically, the vacuum regulators are used to regulate for a specific level of vacuum pressure that is established by the caregiver, which enables the caregiver to adjust the level of suction applied to the patient. To modify the vacuum regulator setting, typically the caregiver manually rotates an actuator or control knob on the vacuum regulator. A vacuum gauge provides a visual indication of the vacuum level.

A common implementation of a medical vacuum/suction system is to suction fluids and particles from the patient during surgery or from a wound site. Suction is applied to the patient (for example, at a wound site). In order to prevent fluids that are suctioned from the patient from contaminating the vacuum regulator or the central vacuum source, a suction canister (also referred to as a suction bottle) is positioned between the patient and the vacuum regulator and in fluid flow communication with the vacuum source. An example of a suction canister is shown in U.S. Pat. No. US11,583,622, issued Feb. 21, 2023. Liquid and large solid particles are gravity-separated from gas by the suction canister, which prevents them from flowing into the vacuum regulator. The internal volume of a typical suction canister is in the range of 700-3000 ml.

For several reasons, contamination of the vacuum regulator and the central vacuum system remain common. First, medical personnel may suction patient fluids without a suction canister in place. Second, the suction canister may overflow, resulting the suctioned liquids and particles bypassing the suction canister and flowing into the vacuum regulator. Third, smaller solid particles (particularly airborne particles) may not be filtered out by the suction canister.

Another issue with conventional medical suction system configurations is that there is no visual indication that fluid is flowing through the system—only that a vacuum pressure is being applied. It is not uncommon for suction tubing to become clogged or blocked (meaning that fluid is no longer being suctioned from the patient). Without a visual indication of flow, there may be a substantial delay in medical personnel becoming aware of a lack of flow/suction.

Efforts have been made to avoid contamination of a vacuum regulator in the event of a suction canister overflow by locating a vacuum trap between the suction cannister and the vacuum regulator. The Vac Trap model PM 9000 by Precision Medical is an example of a vacuum trap. The purpose of a vacuum trap is to stop flow through the vacuum regulator when liquid begins to bypass the suction container and before it reaches the vacuum regulator. A vacuum trap typically consists of a float valve contained within a relatively small chamber (typically less than 300 ml). If liquid begins to bypass the suction container, it will collect on the vacuum trap and the float valve will close, stopping flow to the vacuum regulator.

Although a vacuum trap can avoid contamination of the vacuum regulator and the central vacuum system due to overflow of a suction container (or the failure of medical personnel to use a suction container), contamination of the vacuum regulator and the central vacuum system still occurs from airborne particles that are not filtered by the suction canister or a vacuum trap. Efforts have been made to enhance filtration through the use of an in-line hydrophobic filter, which filters both liquids and airborne particles. However, in-line hydrophobic filters are undesirable because of their relatively high pressure drop, which substantially reduces suction and flow.

Another issue with conventional fluid collection and filtration devices used with hospital-based suction systems is that they are designed to be reusable, which requires them to be disassembled and cleaned. While reusability enables a longer product life, it results in a higher unit cost for the device and requires the device to be taken out of service and cleaned. Many health care providers prefer a lower unit cost discardable device.

Accordingly, there is a need for a relatively low-cost, discardable device that protects vacuum regulators and central vacuum source from contamination by liquids and particles suctioned from patients without substantially degrading flow and suction.

### SUMMARY

A collection canister for use with a vacuum system for withdrawing fluids from a patient (such as at a wound site) is disclosed. The collection canister includes three functional elements in a single discardable housing—a liquid trap, a visual flow indicator, and a non-hydrophobic filter.

In one implementation, the collection canister includes a housing positioned upstream of a vacuum regulator which is fluidly coupled to a vacuum source. A valve positioned within the housing is normally in an open position to permit the withdrawn fluids to collect within the housing. When the fluids reach a predetermined threshold, the valve moves to a closed position to prevent additional fluids from entering the housing. A flow indicator indicates the presence of vacuum flow within the housing. A filter positioned within the housing impedes solids, liquids, and airborne particles from passing through the housing to prevent damage to the vacuum regulator and vacuum source. A mounting adapter assembly to facilitate mounting the collection canister to the vacuum regulator is also disclosed. The following are aspects of the invention.

Aspect 1: An apparatus comprising:
a housing having an inlet port and an outlet port, the housing defining an internal volume;
a liquid trap located within the housing, the liquid trap comprising a reservoir and a float valve; and
a filter comprising a filter media, the filter being located within the housing and in fluid flow communication with the liquid trap;
wherein when fluid flows from the inlet port to the outlet port, the liquid trap is located in upstream fluid flow communication with the filter; and
wherein when fluid flows from the inlet port to the outlet port, all fluid passing through the outlet port is filtered by the filter media before passing through the outlet port.

Aspect 2: The apparatus of aspect 1, wherein the housing comprises a sealed housing.

Aspect 3: The apparatus of any of aspects 1-3, wherein the inlet port is substantially perpendicular to the outlet port.

Aspect 4: The apparatus of aspect 3, wherein the housing comprises a lid, a floor, and a sidewall extending from the lid to the floor, wherein the outlet port is located in the lid and the inlet port is located in the sidewall.

Aspect 5: The apparatus of any of aspects 1-4, further comprising a visual flow indicator adapted to provide a visual indication of flow when fluid flows from the inlet port to the outlet port.

Aspect 6: The apparatus of aspect 5, wherein when fluid flows from the inlet port to the outlet port, the visual flow indicator is located at least partially within a flow conduit that is in upstream fluid flow communication with the liquid trap.

Aspect 7: The apparatus of aspect 6, wherein the housing comprises a lid, wherein the lid has a lid cross sectional area and the flow conduit has a flow conduit cross sectional area that is less than half of the lid cross sectional area.

Aspect 8: An apparatus comprising:
a housing having a lid, a lower floor, an upper floor, a sidewall extending from the lid to the lower floor, a lower chamber extending from the lower floor to the upper floor, an upper chamber extending from the upper floor to the lid, an inlet port extending through the sidewall, and an outlet port extending through the lid;
a flow conduit located at an upper end of the lower chamber, the flow conduit extending through the upper floor and having a flow aperture that provides a fluid flow connection between the lower chamber and the upper chamber;
a float valve located within the lower chamber adapted to prevent fluid flow through the flow conduit when a fluid level in the lower chamber reaches a predetermined threshold and to allow fluid flow through the flow conduit when the fluid level in the lower chamber is below the predetermined threshold;
a flow indicator element positioned at least partially within the flow conduit adapted to provide a visual indication of fluid flow through the flow conduit; and
a filter media positioned between the flow conduit and the outlet port, the filter media and upper chamber being shaped and positioned to force all fluid flow from the flow conduit to the outlet port to pass through the filter media.

Aspect 9: The apparatus of aspect 8, wherein the float valve comprises a buoyant float and a seal located at a lower portion of the flow conduit.

Aspect 10: The apparatus of aspect 9, wherein the float valve further comprises at least one float valve leg located in the lower chamber that restricts lateral movement of the buoyant float within the lower chamber.

Aspect 11: The apparatus of aspect 10, wherein the at least one float valve leg comprises a flow-diverting leg located between the inlet port and the buoyant float.

Aspect 12: The apparatus of aspect 10, wherein each of the at least one float valve leg extends downwardly from the upper floor.

Aspect 13: The apparatus of any of aspects 8-12, wherein the flow conduit has a lower portion that is fully enclosed and an upper portion that is only partially enclosed and comprises a plurality of spaced-apart projections.

Aspect 14: The apparatus of aspect 13, wherein the lower portion is opaque.

Aspect 15: The apparatus of aspect 13, wherein the plurality of spaced-apart projections extend upwardly to a lower side of the filter media.

Aspect 16: The apparatus of any of aspects 8-15, wherein the flow indicator element is disposed within the flow conduit, the flow indicator element being adapted to be located in a seated position within the flow conduit in the absence of fluid flow through the flow conduit and to move vertically upwardly from a seated position in response to the presence of flow through the flow conduit.

Aspect 17: The apparatus of aspect 16, wherein the flow indicator element and lower portion of the flow conduit are configured so that the flow indicator element is contained entirely within the lower portion of the flow conduit when the flow indicator element is in a seated position.

Aspect 18: The apparatus of aspect 17, wherein the flow indicator element is at least partially located in the upper portion of the flow conduit when fluid is flowing through the flow conduit.

Aspect 19: The apparatus of any of aspects 8-18, wherein the flow conduit has a flow conduit diameter and the filter media has a filter media diameter that is at least twice the flow conduit diameter.

Aspect 20: The apparatus of any of aspects 8-19, wherein the filter media is adapted to provide a collection efficiency of at least 99% for a particles size of 5 microns.

Aspect 21: The apparatus of any of aspects 8-20, wherein the lid is permanently affixed to the housing.

Aspect 22: The apparatus of any of aspects 1-21, further comprising:
a mounting adapter having:
   an upper fitting that is male threaded and adapted to be connected to a female threaded inlet fitting on the medical vacuum regulator;
   a lower fitting having an outer surface that is unthreaded;
   an adapter aperture extending through the upper fitting and the lower fitting; and
   at least one engaging flange that is adapted to releasably secure the housing to the adapter.

Aspect 23: The apparatus of aspect 22, wherein the lower fitting comprises a seal seated in a recess located on the outer surface.

Aspect 24: The apparatus of aspect 23, wherein the lid comprises at least one protrusion that engages at least one engaging flange.

Aspect 25: The apparatus of any of aspects 22-24, wherein the housing has a locked position and an unlocked position;
wherein when in the locked position, the outlet port is engaged with the lower fitting, the at least one protrusion is engaged with at least one engaging flange, and the housing cannot be rotated relative to, or removed from, the mounting adapter;
wherein when in the unlocked position, the housing can be removed from the mounting adapter and the outlet port disengaged from the lower fitting.

Aspect 26: The apparatus of aspect 25, wherein the at least one protrusion comprises at least one release tab having a free end.

Aspect 27: The apparatus of any of aspects 22-26, wherein the housing can be changed from the locked position to the unlocked position by deflecting each at least one release tab inwardly.

### BRIEF DESCRIPTION OF THE DRAWINGS

For a more complete understanding of the present invention, reference is made to the following detailed description of embodiments considered in conjunction with the accompanying drawings, in which:

FIG. 1 is a perspective view of a collection canister shown attached to a vacuum regulator;

FIG. 2 is a perspective view of the collection canister;

FIG. 3 is a side elevational view of the collection canister;

FIG. 4 is an exploded view of the collection canister;

FIG. 5 is a perspective view of a mounting adapter for the collection canister, shown affixed to the bottom of the vacuum regulator;

FIG. 6 is a perspective view of the mounting adapter; and

FIG. 7 is an elevational view of the mounting adapter.

### DETAILED DESCRIPTION

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign particular meaning. Further, it should be noted that, as recited in the specification and in the claims appended hereto, the singular forms “a,” “an,” and “the” include the plural referents unless otherwise stated. Additionally, the terms “comprises” and “comprising” when used herein specify that certain features are present in that embodiment but should not be interpreted to preclude the presence or addition of additional features, components, operations, and/or groups thereof.

The following disclosure is intended to be read in connection with the accompanying drawings, which are to be considered part of the entire written description of the invention. The drawing figures are not necessarily to scale and certain features of the invention may be shown exaggerated in scale or in somewhat schematic form in the interest of clarity and conciseness. In this description, relative terms such as “horizontal,” “vertical,” “up,” “down,” “top,” “bottom,” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both moveable or rigid attachments or relationships, unless expressly described otherwise, and includes terms such as “directly” coupled, secured, etc. The term “operatively coupled” is such an attachment, coupling, or connection that allows the pertinent structures to operate as intended by virtue of that relationship.

As used herein, the term “collection efficiency” means the percentage of the specified particle size that is filtered out by the filter media using the following test methodology. An aerosol generator is used to provide an upstream concentration of at least 1×10⁷ particles per liter for the particle size in question. Particle size means the diameter of the particle. Fluid from the aerosol generator is flowed through the filter media (having an area of 100 cm²) at a face velocity of 5.3 cm/sec for a period of 1 minute. Upstream and downstream particle concentrations are measured using a laser particle counter. Collection efficiency is equal to (1−(measured upstream particle concentration/measured downstream particle concentration))×100. The test is conducted at a temperature between 20 and 30 degrees C. and relative humidity between 45 and 85%.

As used herein, the term “sealed housing” means that the housing is either created out of a single piece of material or is created with multiple elements that, once assembled, cannot be disassembled without damaging the housing. Non-exclusive assembly methods for elements of a sealed housing include welding (laser, ultrasonic, infrared, induction, etc.), adhesives, solvent cementing, and heat staking.

As used herein, the term “non-hydrophobic filter media” means a filter media that is not designed to repel water and is not made from, or treated with, a water-repelling substances, such as polytetrafluoroethylene (PTFE), polypropylene, or another hydrophobic polymer.

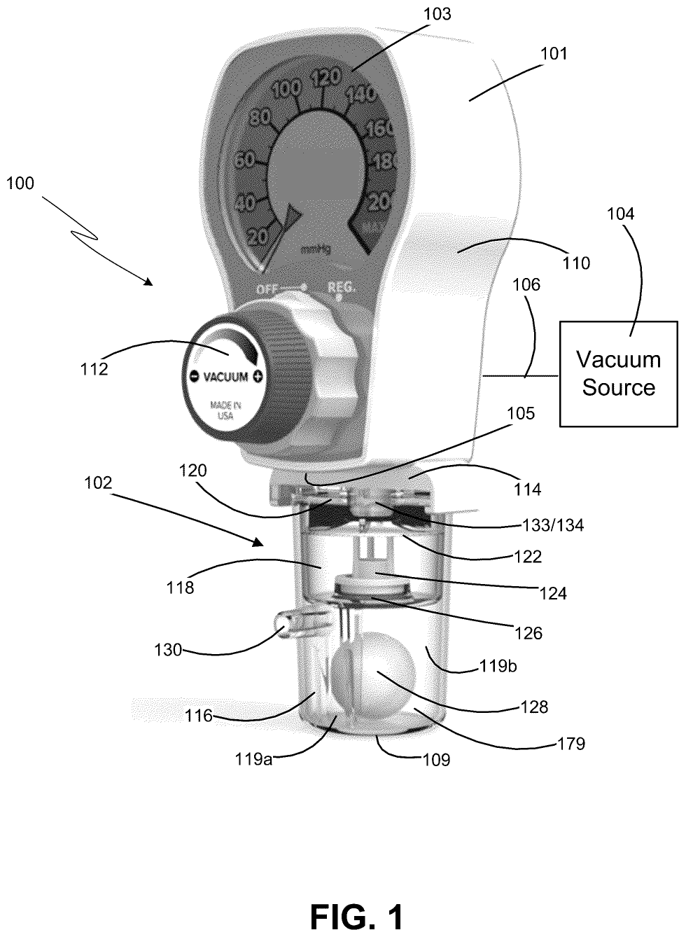

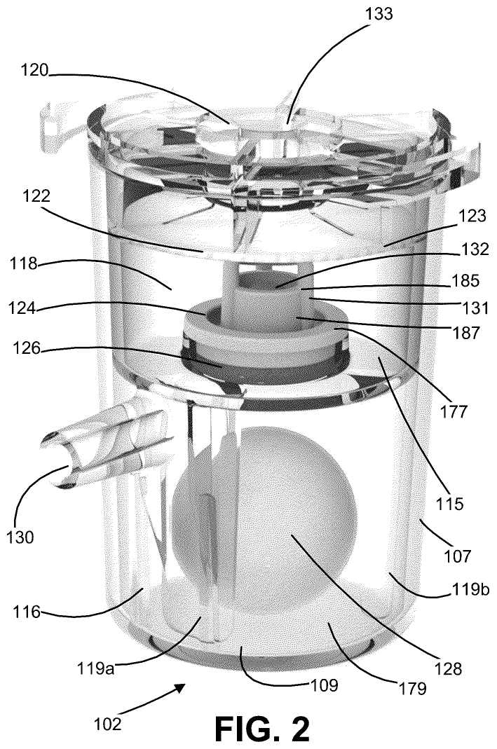

As shown in FIGS. 1-4, an exemplary implementation of a multi-function collection canister 102 for use as part of a vacuum withdrawal system 100 is shown. The vacuum withdrawal system 100 includes a vacuum regulator 110 that connects to an external vacuum source 104 (shown schematically). The external vacuum source 104 may include a central vacuum source of a healthcare facility, central vacuum pump remotely located from the suction regulator, or a separate portable vacuum pump. Such a connection may be made via an appropriately configured coupler 106 located on the rear of the vacuum regulator 110. The system 100 may be used for numerous health provider procedures and devices. The central vacuum source may be plumbed into the building structure of a healthcare facility. “Healthcare facility” is intended to include hospitals, outpatient treatment facilities, doctors'offices, nursing homes, and any other facility in which healthcare services are provided.

The vacuum regulator 110 is attached upstream of the external vacuum source 104 to provide a regulated source of vacuum flow to the patient. As used herein, the source of vacuum flow will be at a downstream location and the patient will be at an upstream location, such that the terms “upstream” and “downstream” will reference the flow of fluid in the direction from the patient toward the central vacuum source.

The vacuum regulator 110 includes a housing 101 and is provided with a rotatable control knob 112 mounted to the housing 101 to enable the caregiver to modify the level of vacuum flow or negative pressure applied to the patient. A vacuum gauge 103 positioned on the housing 101 above the control knob 112 provides a visual indication of the vacuum pressure. The vacuum regulator 110 may also include an inlet stem 105 (FIG. 1) located at the bottom center of the housing 101 to enable fluid communication between the vacuum regulator 110 and the multi-function collection canister 102. The inlet stem 105 may be a cylindrical through opening that is internally threaded. The vacuum regulator 110 may be used in a variety of applications for regulating pressure for procedures such as but not limited to: nasopharyngeal, tracheal, surgical, gastrointestinal, pleural, wound drainage, etc.

A collection canister 102 is shown coupled to and in fluid communication with the vacuum regulator 110. The collection canister 102 includes a housing 116 having a side wall 107 integral with and extending upwardly from a lower floor 109, and an open top 111. In this implementation, the side wall 107 is generally cylindrical in shape, with a slight taper from the top 111 to the lower floor 109. The housing 116 defines an internal volume which includes a lower chamber 179 and an upper chamber 129 located above the lower chamber 179. The lower chamber 179 extends from the lower floor 109 to an upper floor 115 and includes a lower chamber height extending from the lower floor 109 to the upper floor 115. The upper chamber 129 extends from the upper floor 115 to a lid 120, which is arranged to be secured to the housing 116. The lid 120 may be permanently affixed to the housing 116, such that removal of the lid 120 cannot be affected without breaking or otherwise destroying the housing 116. The upper chamber 129 may include a regulator-engaging aperture 181 that extends through the lid 120 and into the upper chamber 129. The lower chamber 179 may include a suction-tube engaging aperture 183 that extends through the side wall or side wall 107 into the lower chamber 179. The housing 116 may include an inlet port 130 arranged to connect to a suction device (not shown), such as a cannula, a catheter, a tube or other device, which is introduced to a location on the patient in which fluids and tissue are to be suctioned (e.g., a wound site). The housing 116 may be formed of any suitable material, e.g., clear high-impact styrene.

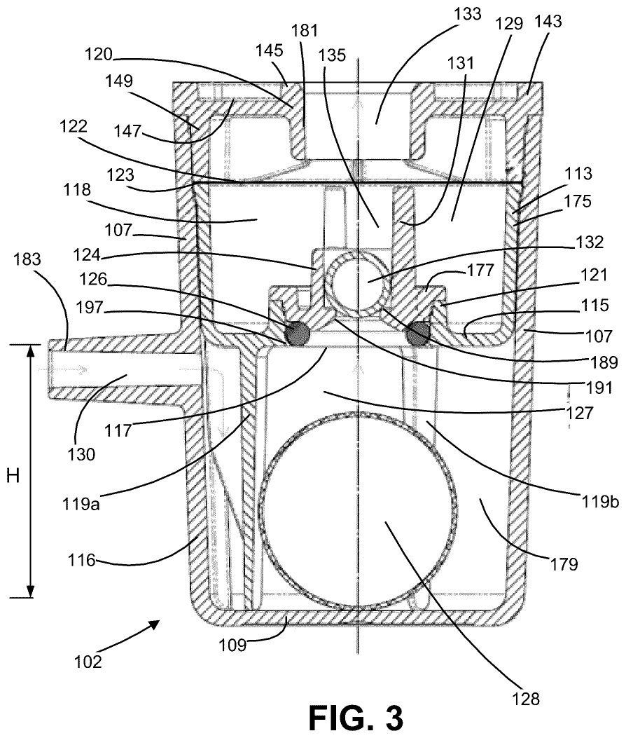

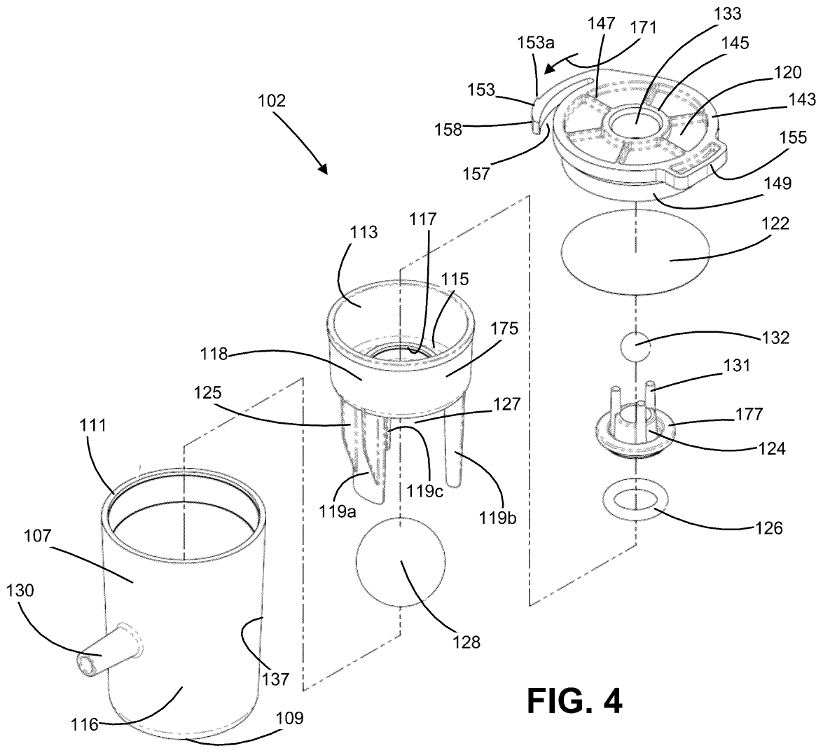

The collection canister 102 includes a cage 118 arranged to fit snugly within the side wall 107 of the housing 116. The cage 118 may be formed of any suitable material, e.g., clear high-impact styrene, and includes an upper portion 113 formed of a cylindrical sidewall 175 and the upper floor 115, which includes a central opening 117. The cylindrical sidewall 175 extends upwardly from the circumference of the upper floor 115 to enable the snug fit within the housing 116. As best shown in FIG. 3, the cage 118 includes a shorter annular flange 121 extending upwardly from the central opening 117. The cage 118 includes a plurality of legs 119a, 119b, and 119c, e.g., three legs, extending downwardly from the circular floor 115 of the cage 118 to contact the lower floor 109 of the housing 116. As best shown in FIG. 4, the legs 119a, 119b, and 119c may include reinforcement ribs 125. When disposed within the lower chamber 179 of the housing 116, the legs 119a, 119b, and 119c form a vertically oriented guidance channel 127 in which a float 128 is confined. In addition, in this implementation, leg 119a is positioned between the inlet port 130 and the float 128, and thereby provides a flow-diverting function that restricts lateral movement of the buoyant float 128 that would otherwise be caused by flow through the inlet port 130. Together, the guidance channel 127 and buoyant float 128 form a shut-off valve (e.g., a float valve). In this way, the lower chamber 179 of the housing 116 forms a liquid trap comprising a reservoir which retains liquid and the float valve which prevents liquid from entering the upper chamber 129 of the housing 116.

As the vacuum regulator 110 is adjusted, a desired level of vacuum flow is transmitted from the vacuum source to the suction device to withdraw fluids from the patient. Withdrawn fluids, either liquid or gas, travel downstream from the patient wound site through the suction device, and are received and collected through the inlet port 130 and into a lower chamber 179 of the housing 116 where the guidance channel 127 is located. The float 128, e.g., a polypropylene ball, rises within the guidance channel 127 as the liquid level inside the housing 116 rises. As the liquid level rises towards the top of the guidance channel 127, the float 128 presses against the central opening 117 of the cage 118 to close off the central opening 117. To ensure a tight seal between the float 128 and the central opening 117, a gasket in the form of an O-ring or seal 126 may be provided at a lower end of the central opening 117, on a lower side 197 of the upper floor 115. The O-ring or seal 126 is adapted to prevent fluid flow communication from the lower chamber 179 to the upper chamber 129 when the float 128 is engaged with the O-ring or seal 126. The O-ring or seal 126 may be formed of any suitable material, e.g., silicone. The housing 116 and the cage 118 may be made from polymers for light weight and impact resistance.

A flow indicator or flow conduit 124 is interposed within the collection canister 102 to provide the caregiver with a visual indicator of relative flow within the vacuum withdrawal system 100, e.g., no flow, low flow, intermediate flow, and high flow conditions, as well as to determine whether there is a blockage in vacuum flow. The flow conduit 124 may be formed of any suitable material, e.g., high-impact styrene. As best shown in FIG. 3, the flow conduit 124 is situated within an upper chamber 129 of the collection canister 102 and is disposed on the annular flange 121 of the cage 118. The flow conduit 124 may be attached to the upper floor 115. The flow conduit 124 includes a fully enclosed lower portion comprising a cylindrical flow housing sidewall 187, a flow housing upper end 185, and a partially enclosed upper portion comprising a plurality of ribs or spaced-apart projections 131. The cylindrical flow housing sidewall 187 defines a flow aperture 191 that extends through the upper floor 115 and enables fluid flow communication from the lower chamber 179 to the upper chamber 129. The plurality of spaced-apart projections 131 extend upwardly from the cylindrical flow housing sidewall 187 and a base portion or lower end 177 to form an open guidance channel 135 permitting the flow of gaseous materials therethrough. The shut-off valve (e.g., the float valve) is formed by the buoyant float 128 and the O-ring or seal 126 located at a lower portion of the flow conduit 124. In order to effectively provide a visual indication of flow, the flow conduit 124 may have a cross sectional area that is less than half of the cross sectional area of the canister lid 120 (which is representative of the overall cross-sectional area of the housing 101). The lower portion of the flow conduit 124 may be opaque.

The cylindrical flow housing sidewall 187 and the plurality of spaced-apart projections 131 may be adapted to retain a visual flow indicator 132, such as a ball, for example. The visual flow indicator 132 is able to move from the flow housing lower end 177 to the flow housing upper end 185. The flow housing lower end 177 may include a retaining flange 189 adapted to prevent the visual flow indicator 132 from moving below the retaining flange 189. The visual flow indicator 132 is adapted to be located in a seated position within the flow conduit 124 in the absence of fluid flow through the flow conduit 124 and to move vertically upwardly from a seated position in response to the presence of flow through the flow conduit. The visual flow indicator 132 and the flow conduit 124 may also be configured so that the visual flow indicator 132 is contained entirely within the lower portion of the flow conduit 124 when the visual flow indicator 132 is in a seated position. The visual flow indicator 132 may be formed of any suitable material, e.g., polypropylene. The visual flow indicator 132 is positioned within the guidance channel 135 and is captured by a filter media 122 positioned over the top ends of the ribs or spaced-apart projections 131. In this manner, as the vacuum flow passes through the fluid pathway, the visual flow indicator 132 moves upwardly from a seated position, such that the visual flow indicator 132 is located at least partially within the flow conduit 124, indicating the presence of flow therethrough. As best shown in FIG. 3, once exiting the flow conduit 124, the vacuum flow disperses evenly across the surface area of the filter media 122 for optimal filtering. In other implementations, the visual flow indicator 132 could be a different shape, such as a cylindrical shape. In yet other implementations, the visual flow indicator 132 could be another type of visual flow indicator, such as an impeller, where its rotation provides the visual indication of flow, or a flapper, where its rotational displacement provides the visual indication of flow.

The filter media 122 may be located between the flow housing upper end 185 and the regulator-engaging aperture 181. The filter media 122 is generally disc-shaped and includes a perimeter 123 that extends to and conforms within the cylindrical sidewall 175 of the cage 118 and may be retained in place within the upper chamber 129 of the housing 116 between the cylindrical flange 149 of the canister lid 120 and the cylindrical sidewall 175 of the cage 118. The filter media 122 extends to the sidewall 175 along the entire perimeter 123. The filter media 122 is permeable by gas only and impedes solids, liquids, or airborne particles that may be interposed in the vacuum flow to prevent them from being introduced into the vacuum regulator 110 or the central vacuum source. The filter media 122 may be a porous polymer that impedes solids, liquids, and airborne particles from passing but allows gaseous materials within the vacuum flow to pass through the filter media 122 and exit the collection canister 102 through the central filter opening 133 of the canister lid 120. The filter media 122 may be formed from one or more polymers and/or other natural substances. In this exemplary implementation, the filter media 122 may be a media that provides a collection efficiency of at least 99% at a particle size of 5 microns. In another implementation, the filter media 122 may be a media that provides a collection efficiency of at least 99% at a particle size of 1 micron. In yet another implementation, the filter media 122 may be a media that provides a collection efficiency of at least 99% at a particle size of 0.2 microns.

The canister lid 120 is formed of an annular ring 143 and a hub 145 positioned centrally within the annular ring 143, the hub 145 being connected to the annular ring 143 by a plurality of spokes 147 branching therebetween. A cylindrical flange 149 is shown extending downwardly from the annular ring 143, the cylindrical flange 149 being sized to conform with the inner surface 137 of the side wall 107 of the housing 116 to provide a snug fit of the canister lid 120 within the housing 116. The canister lid 120 may be affixed to the housing 116 by any suitable method, e.g., ultrasonic welding. The filter opening 133 serves as an outlet port to enable fluid communication with the vacuum regulator 110 when the collection canister 102 is coupled to it. In this manner, vacuum flow exiting the filter media 122 may flow through the filter opening 133 and into the vacuum regulator 110 devoid of solids, liquids, and airborne particles to prevent contamination of the vacuum regulator and vacuum source. The canister lid 120 may be formed of any suitable material, e.g., high-impact styrene.

In this implementation, the inlet port 130 is located in the side wall 107, and therefore, is substantially perpendicular to the outlet port (defined by filter opening 133). The inlet port 130 is also preferably located distal to the lower floor 109 and just below the upper floor 115. In other words, the inlet port 130 is preferably located in the upper ¼ of the height H of the lower chamber 179. The configuration enhances the liquid trapping functionality of the collection canister 102.

The canister lid 120 may be provided with a locking mechanism to secure the collection canister 102 to a mounting adapter 114, the locking mechanism including an actuatable release tab 153 and a protrusion 155 located at opposite ends of the annular ring 143 of the canister lid 120. The actuatable release tab 153 may include a free end 158 that extends at an arc and exterior to the annular ring 143 to provide a gap area 157 between the release tab 153 and the annular ring 143 such that in response to actuation, the release tab 153 deflects or moves into the gap area 157. The release tab 153 may also include a stop surface 153a on its exterior surface.

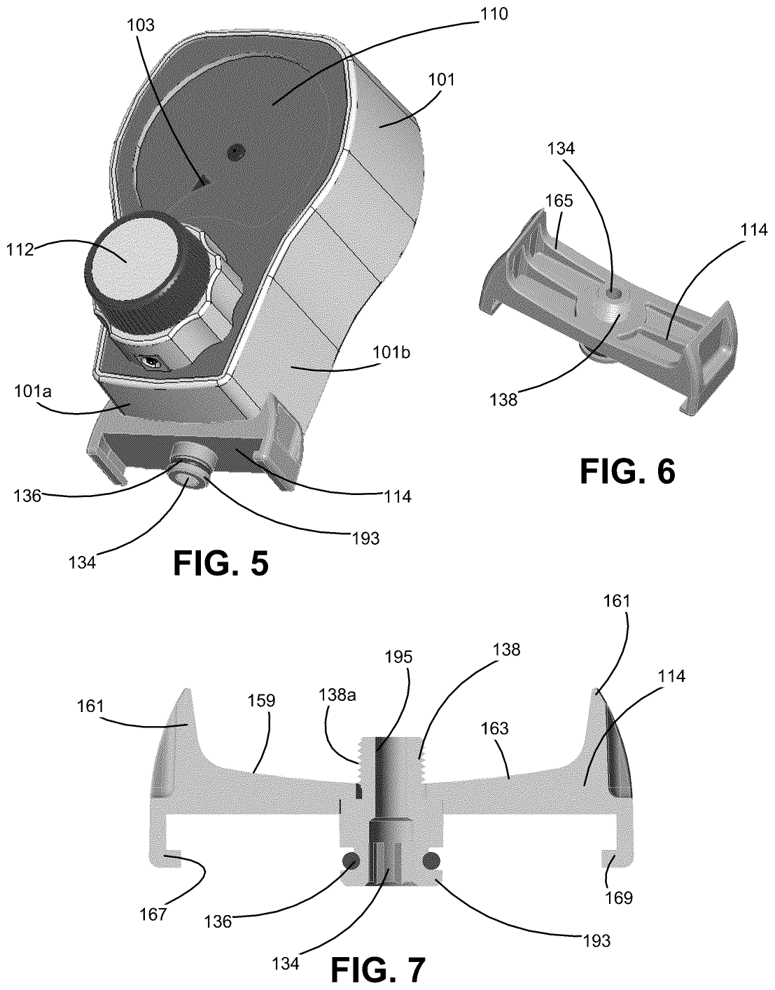

Referring now to FIGS. 5-7, the mounting adapter 114 is an elongate member having an upward facing surface 159 and wings 161 extending upwardly at opposite ends thereof. The wings 161 are adapted to releasably secure the housing 101 of the vacuum regulator 110 to the mounting adapter 114. Together, the upward facing surface 159 and wings 161 form an engagement surface 163 having a contour that is complimentary to the bottom surface 101a and side surface 101b of the vacuum regulator housing 101 (FIG. 5). The engagement surface 163 may include ribs 165 to provide added gripping capability. The mounting adapter 114 also includes a central hub or male fitting 138 that aligns with the inlet stem 105 of the vacuum regulator 110 when the vacuum regulator 110 is positioned within the engagement surface 163. The male fitting 138 includes an external male thread 138a operatively arranged to threadedly engage with and connect to the female threaded inlet fitting or inlet stem 105 of the vacuum regulator 110 to secure the mounting adapter 114 at the lower end of the vacuum regulator housing 101. The mounting adapter 114 may also include a lower fitting 193 having an outer surface that is unthreaded. An adapter aperture 195 extends through the upper male fitting 138 and the lower fitting 193. The adapter aperture 195 defines a central vacuum opening 134 to provide fluid communication into the vacuum regulator 110. The lower fitting 193 may be provided with an O-ring or seal 136 to ensure a tight seal between the adapter and the collection canister 102. The mounting adapter 114 includes a first hook or first engagement flange 167 and a second hook or second engagement flange 169 that extend downwardly from opposite ends thereof, with the first engagement flange 167 opposing the second engagement flange 169. The mounting adapter 114 may also include a lower fitting having an outer surface that is unthreaded, and a seal seated in a recess located on the outer surface.

The housing 116 of the collection canister 102 has a locked position and an unlocked position. When in the locked position, the outlet port formed by the filter opening 133 is engaged with the lower fitting 193 of the mounting adapter 114, the protrusion of the cannister lid 120 is engaged with the second engagement flange 169 of the mounting adapter, and the housing 116 cannot be rotated relative to, or removed from, the mounting adapter. When in the unlocked position, the housing 116 can be removed from the mounting adapter 114 and the outlet port formed by the filter opening 133 disengaged from the lower fitting 193.

The mounting adapter 114 may be positioned over the canister lid 120 and rotated in a first direction 171 to bring the first engagement flange 167 into engagement with the stop surface 153a and to bring the second engagement flange 169 into engagement with the protrusion 155 to releasably attach the mounting adapter 114 to the collection canister 102 in the locked position. With the vacuum regulator 110 mounted to the collection canister 102, fluids may be withdrawn from the patient site and collected in the collection canister 102. Once collected fluids have reached a predetermined threshold, the caregiver may actuate or press the release tab 153 into the gap area 157 (i.e., deflect the release tab 153 inwardly) to unlock the housing 116, and rotate the collection canister 102 in a direction opposite to the first direction 171 to release the collection canister 102 from the mounting adapter 114 for disposal and replacement of the collection canister 102.

The mounting adapter 114 can be an integral part of the vacuum regulator 110 or can be provided as an accessory or component with the vacuum regulator 110. Alternatively, the mounting adapter 114 may be provided as a stand-alone accessory intended to be retrofitted to an existing regulator.

As such, an invention has been disclosed in terms of preferred embodiments and alternate embodiments thereof. Of course, various changes, modifications, and alterations from the teachings of the present invention may be contemplated by those skilled in the art without departing from the intended spirit and scope thereof. It is intended that the present invention only be limited by the terms of the appended claims.

1. An apparatus comprising:
a housing having an inlet port and an outlet port, the housing defining an internal volume;
a liquid trap located within the housing, the liquid trap comprising a reservoir and a float valve; and
a filter comprising a filter media, the filter being located within the housing and in fluid flow communication with the liquid trap;
wherein when fluid flows from the inlet port to the outlet port, the liquid trap is located in upstream fluid flow communication with the filter; and
wherein when fluid flows from the inlet port to the outlet port, all fluid passing through the outlet port is filtered by the filter media before passing through the outlet port.

2. The apparatus of claim 1, wherein the housing comprises a sealed housing.

3. The apparatus of claim 1, wherein the inlet port is substantially perpendicular to the outlet port.

4. The apparatus of claim 3, wherein the housing comprises a lid, a floor, and a sidewall extending from the lid to the floor, wherein the outlet port is located in the lid and the inlet port is located in the sidewall.

5. The apparatus of claim 1, further comprising a visual flow indicator adapted to provide a visual indication of flow when fluid flows from the inlet port to the outlet port.

6. The apparatus of claim 5, wherein when fluid flows from the inlet port to the outlet port, the visual flow indicator is located at least partially within a flow conduit that is in upstream fluid flow communication with the liquid trap.

7. The apparatus of claim 6, wherein the housing comprises a lid, wherein the lid has a lid cross sectional area and the flow conduit has a flow conduit cross sectional area that is less than half of the lid cross sectional area.

8. An apparatus comprising:
a housing having a lid, a lower floor, an upper floor, a sidewall extending from the lid to the lower floor, a lower chamber extending from the lower floor to the upper floor, an upper chamber extending from the upper floor to the lid, an inlet port extending through the sidewall, and an outlet port extending through the lid;
a flow conduit located at an upper end of the lower chamber, the flow conduit extending through the upper floor and having a flow aperture that provides a fluid flow connection between the lower chamber and the upper chamber;
a float valve located within the lower chamber adapted to prevent fluid flow through the flow conduit when a fluid level in the lower chamber reaches a predetermined threshold and to allow fluid flow through the flow conduit when the fluid level in the lower chamber is below the predetermined threshold;
a flow indicator element positioned at least partially within the flow conduit adapted to provide a visual indication of fluid flow through the flow conduit; and
a filter media positioned between the flow conduit and the outlet port, the filter media and upper chamber being shaped and positioned to force all fluid flow from the flow conduit to the outlet port to pass through the filter media.

9. The apparatus of claim 8, wherein the float valve comprises a buoyant float and a seal located at a lower portion of the flow conduit.

10. The apparatus of claim 9, wherein the float valve further comprises at least one float valve leg located in the lower chamber that restricts lateral movement of the buoyant float within the lower chamber.

11. The apparatus of claim 10, wherein the at least one float valve leg comprises a flow-diverting leg located between the inlet port and the buoyant float.

12. The apparatus of claim 10, wherein each of the at least one float valve leg extends downwardly from the upper floor.

13. The apparatus of claim 8, wherein the flow conduit has a lower portion that is fully enclosed and an upper portion that is only partially enclosed and comprises a plurality of spaced-apart projections.

14. The apparatus of claim 13, wherein the lower portion is opaque.

15. The apparatus of claim 13, wherein the plurality of spaced-apart projections extend upwardly to a lower side of the filter media.

16. The apparatus of claim 8, wherein the flow indicator element is disposed within the flow conduit, the flow indicator element being adapted to be located in a seated position within the flow conduit in the absence of fluid flow through the flow conduit and to move vertically upwardly from a seated position in response to the presence of flow through the flow conduit.

17. The apparatus of claim 16, wherein the flow indicator element and lower portion of the flow conduit are configured so that the flow indicator element is contained entirely within the lower portion of the flow conduit when the flow indicator element is in a seated position.

18. The apparatus of claim 17, wherein the flow indicator element is at least partially located in an upper portion of the flow conduit when fluid is flowing through the flow conduit.

19. (canceled)

20. The apparatus of claim 8, wherein the filter media is adapted to provide a collection efficiency of at least 99% for a particle size of 5 microns.

21. The apparatus of claim 8, wherein the lid is permanently affixed to the housing.

22.-27. (canceled)
